(19) United States
(12) Patent Application Publication
(10) Pub. No.: US 2009/0187040 A1
(43) Pub. Date: Jul. 23, 2009

(54) FENOFIBRIC ACID POLYMORPHS; METHODS OF MAKING; AND METHODS OF USE THEREOF

(76) Inventors: Tong Sun, Marlton, NJ (US); Michael L. Hall, Albany, NY (US); Paul K. Isbester, Castleton, NY (US); Brent R. Whitehead, Piscataway, NJ (US)

Correspondence Address: CANTOR COLBURN, LLP, 20 Church Street, 22nd Floor, Hartford, CT, 06103, US

(21) Appl. No.: 12/354,043

(22) Filed: Jan. 15, 2009

Related U.S. Application Data
(60) Provisional application No. 61/021,993, filed on Jan. 18, 2008.

(51) Int. Cl. C07C 69/76

(52) U.S. Cl. 560/52

(57) ABSTRACT

Disclosed are new methods of making the fenofibric acid polymorphs as well as formulations prepared therefrom and uses thereof.

### CROSS REFERENCE TO RELATED APPLICATION

This application claims the benefit of U.S. Provisional Application Ser. No. 61/021,993 filed Jan. 18, 2008, which is hereby incorporated by reference in its entirety.

### BACKGROUND

Fenofibrate, 2-[4-(4-chlorobenzoyl)phenoxy]-2-methyl-propanoic acid, 1-methylethyl ester, is used in the treatment of endogenous hyperlipidaemias, hypercholesterolaemias and hypertriglyceridaemias in adults. Fenofibric acid, the active metabolite of fenofibrate, produces reductions in total cholesterol, LDL cholesterol, apolipoprotein B, total triglycerides and triglyceride rich lipoprotein (VLDL) in treated patients. Also, treatment with fenofibrate results in increases in high-density lipoprotein (HDL) and apoproteins apoAI and apoAII. Prolonged treatment with fenofibrate at the rate of about 300 to about 400 mg per day makes it possible to obtain a reduction in total cholesterol of about 20 to about 25% and a reduction in the levels of triglycerides of about 40 to about 50%.

Fenofibrate is not soluble in water, which limits its absorption in the gastrointestinal (GI) tract. To remedy this problem, research groups have tried a multitude of strategies including, for example, formulations comprising reduced sized fenofibrate, the combination of fenofibrate and vitamin E, the use of diethylene glycol monoethyl ether (DGME) as solubilizer, and the combination of fenofibrate with one or more polyglycolyzed glycerides.

Polymorphs are solid crystalline phases of an active agent differing by the arrangement of the active agent molecules in the solid state. Different polymorphs of the same active agent can exhibit different physical properties such as solubilities, melting points, hardness, optical properties, dissolution, and the like. Differences in the dissolution of the polymorphs can result in differences in the therapeutic activity between the different polymorphs.

Polymorphism is an important consideration in formulating an active agent, specifically in regard to solubility of the active agent and dissolution from a dosage formulation. Use of a particular polymorph may provide superior solubility, dissolution, and possibly increased bioavailability.

There remains a need in the art for improved processes of preparing fenofibric acid polymorphic forms.

### SUMMARY

In one embodiment, a method of preparing fenofibric acid Form B comprises crystallizing fenofibric acid from a crystallization solution comprising fenofibric acid and a solvent system comprising i) ethyl acetate; ii) methyl ethyl ketone; iii) ethyl acetate as a primary solvent and water as an anti-solvent; iv) dioxane as a primary solvent and heptane as an anti-solvent; or v) tert-amyl alcohol.

In another embodiment, a method of preparing fenofibric acid Form A comprises crystallizing fenofibric acid from a crystallization solution comprising fenofibric acid and a solvent system comprising i) dichloromethane; ii) methanol; iii) acetone; iv) dichloromethane as a primary solvent and water, heptane, or c-hexane as an anti-solvent; v) benzene, toluene, or xylenes as a primary solvent and heptane or c-hexane as an anti-solvent; v) 1,2-dimethoxyethane, isopropanol, dioxane, dimethylacetamide, methanol, ethanol, or acetonitrile as a primary solvent and water as an anti-solvent; or vi) methanol, dimethyl sulfoxide, or dimethylacetamide as a primary solvent and heptane as an anti-solvent.

In yet another embodiment, a method of preparing fenofibric acid Form B comprises slurrying fenofibric acid Form A seeded with fenofibric acid Form B in acetonitrile at about 15° C. to about 40° C.; or slurrying a mixture of Form A and Form B in water, toluene, water/isopropyl alcohol, dichloromethane, or acetonitrile at about 15° C. to about 40° C.

In still yet another embodiment, a method of preparing fenofibric acid Form A comprises slurrying fenofibric acid Form B in water, acetonitrile, or 1:1 water/isopropyl alcohol at about 30° C. to about 50° C.; or slurrying a mixture of Form A and Form B in water, toluene, 1:1 water/isopropyl alcohol, dichloromethane, or acetonitrile at about 30° C. to about 50° C.

In one embodiment, a method of preparing fenofibric acid Form A comprises lyophilizing a mixture of fenofibric acid and water or a mixture of fenofibric acid, water and methanol.

In another embodiment, a method of preparing fenofibric acid Form B comprises lyophilizing a mixture of fenofibric acid, water, and acetonitrile.

In yet another embodiment, a method of preparing fenofibric acid Form A comprises grinding fenofibric acid Form B or a combination comprising fenofibric acid Form B and Form A under high shear conditions.

In one embodiment, a method of preparing fenofibric acid Form A or Form B comprises preparing fenofibric acid Form A by vapor diffusion from dioxane;heptane, acetone:water and dimethylacetamide:water or Form B by vapor diffusion from dichloromethane:cyclohexane.

These and other embodiments, advantages and features of the present invention become clear when detailed description and examples are provided in subsequent sections.

### DETAILED DESCRIPTION

Disclosed herein are novel methods of preparing two polymorphs of fenofibric acid (2-[4-(4-chlorobenzoyl)phenoxy]-2-methyl-propanoic acid) described herein as Form A and Form B. The two forms were found to be enantiotropically related with Form B being more thermodynamically stable at lower temperatures (e.g., room temperature) and Form A being more stable at elevated temperatures (e.g., above about 45° C.).

The polymorphic forms of fenofibric acid can be prepared using a variety of techniques including crystallization from solution, lyophilization, vapor diffusion, slurrying, or grinding.

New processes of preparing fenofibric acid Form A and Form B include crystallization from a solvent system containing a single solvent or two or more solvents. Optionally, an anti-solvent can be used.

In a generalized procedure, fenofibric acid is dissolved in a solvent system with optional heating to form a crystallization solution. The heated solution can be at about the boiling point of the solvent system, specifically about 25 to about 100° C., more specifically about 30 to about 90° C., yet more specifically about 40 to about 80° C., and still yet more specifically about 50 to about 70° C.

The crystallization solution can be allowed to stand at ambient temperature or cooled to a lower temperature to allow crystal formation. Temperatures for crystal formation can be about −20 to about 25° C., specifically about −10 to about 20° C., more specifically about 0 to about 15° C., and yet more specifically about 3 to about 10° C.

The crystallization can be accomplished with slow cooling or rapid cooling. Rapid cooling can involve placing the crystallization solution under conditions of the targeted lower temperature without a gradual lowering of the temperature. Slow cooling can involve reducing the temperature of the crystallization solution at about 1 to about 30° C. per hour, specifically about 5 to about 25° C. per hour, and yet more specifically about 10 to about 20° C. per hour to a targeted lower temperature.

Optionally, the crystallization solution, prior to any solids formation, can be filtered to remove any undissolved solids, solid impurities and the like prior to removal of the solvent. Any filtration system and filtration techniques known in the art can be used.

In one embodiment, the crystallization solutions can be seeded with the desired polymorph Form A or B.

Suitable solvents for preparing the crystalline forms of fenofibric acid include those that do not adversely affect the stability of the fenofibric acid, and are preferably inert. Suitable solvents may be organic, aqueous, or a mixture thereof Suitable organic solvents may be aliphatic alcohols such as methanol (MeOH), ethanol (EtOH), n-propanol, isopropanol (IPA), n-butanol, tert-amyl alcohol (t-AmOH); ethers such as tetrahydrofuran (THF), dioxane, methyl-tert-butyl ether, 1,2-dimethoxyethane (DME), and 2-methyl tetrahydrofuran; aliphatic ketones such as acetone, methyl ethyl ketone (MEK), and methyl isobutyl ketone; aliphatic carboxylic esters such as methyl acetate, ethyl acetate (EtOAc), and isopropyl acetate; aromatic hydrocarbons such as benzene, toluene, and xylene; aliphatic hydrocarbons such as hexane; aliphatic nitriles such as acetonitrile (MeCN); chlorinated hydrocarbons such as dichloromethane (DCM), chloroform, and carbon tetrachloride; aliphatic sulfoxides such as dimethyl sulfoxide (DMSO); amides such as dimethylformamide (DMF) and dimethylacetamide (DMA); organic acids such as acetic acid; N-methyl-2-pyrrolidone; pyridine; and the like, as well as mixtures comprising at least one of the foregoing organic solvents. Other solvents can be used as an anti-solvent to induce crystal formation of the fenofibric acid from solution. Exemplary anti-solvents include those solvents for which fenofibric acid is not readily soluble in, such as water, heptanes, c-hexane, and the like, and combinations thereof.

“Solvent system” means a single or a combination of two or more solvents.

In one embodiment, fenofibric acid Form B substantially free of Form A is prepared by crystallizing fenofibric acid from a solution of ethyl acetate or methyl ethyl ketone as the primary solvent.

In one embodiment, fenofibric acid Form A substantially free of Form B is prepared by crystallizing fenofibric acid from a solution of dichloromethane.

In one embodiment, the crystallization solution to prepare either Form A or Form B is not an acidified solution (e.g., acidified with hydrochloric acid, hydrogen chloride, etc.).

Lyophilization of solutions containing fenofibric acid can afford Form A, Form B, or a combination of both forms depending upon the solvent system employed. For example, Form A can be prepared by lyophilization from water; Form B can be prepared by lyophilization using water:acetonitrile ratios from about 10:90 to about 95:5, more specifically about 50:50 to about 85:15, yet more specifically about 60:40 to about 80:20, and still yet more specifically about 65:35 to about 75:25. A mixture of Form A and Form B can be prepared by lyophilization using water:isopropyl alcohol ratios from about 10:90 to about 95:5, more specifically about 85:15 to about 95:5. Suitable lyophilization techniques known in the art can be used.

In another embodiment, fenofibric acid Form A can be prepared by intense grinding of fenofibric acid Form B, e.g., use of a ball mill, jet mill, impact mill, hammer mill, and the like. Optionally, the grinding can be performed with heating.

Either polymorph of fenofibric acid can be prepared via vapor diffusion techniques using a primary solvent and an anti-solvent. Vapor diffusion using dioxane as a primary solvent and heptanes as the antisolvent results in the formation of Form A. Use of either acetone or dimethyl acetamide as the primary solvent and water as the anti-solvent also results in Form A. Vapor diffusion using dichloromethane as the primary solvent and c-hexane as the anti-solvent results in the formation of fenofibric acid Form B.

Slurrying, as opposed to complete dissolution of fenofibric acid in a solvent system, can be used to convert one form to another. For example Form B can be converted to Form A by slurrying in water, acetonitrile, or 1:1 water/isopropyl alcohol above ambient temperature but below the temperature at which the fenofibric acid decomposes, specifically above 35° C. Form B can be converted to a combination of Form A and Form B by slurrying in dichloromethane at elevated temperatures disclosed above. Form A can be converted to a combination of Form A and Form B by slurrying in dichloromethane at elevated temperatures disclosed above. The slurrying can be accomplished over several days or weeks, specifically less than 1 day to about 21 days, more specifically about 3 days to about 14 days, and yet more specifically about 5 days to about 7 days.

A 1:1 mixture of Form A and Form B can be converted to substantially all Form B by slurrying the combination in water, toluene, 1:1 water/isopropyl alcohol, dichloromethane, or acetonitrile below about 50° C., specifically below about 25° C. (e.g., from about 0 to about 50° C., specifically about 10 to about 30° C.). A 1:1 mixture of Form A and Form B can be converted to substantially all Form A by slurrying the combination in water, toluene, 1:1 water/isopropyl alcohol, dichloromethane, or acetonitrile at elevated temperatures as disclosed above.

In one embodiment, fenofibric acid Form A in acetonitrile and seeded with Form B is slurried for about 7 to about 10 days to afford fenofibric acid Form B, specifically about 8 to about 9 days. The slurrying is performed at about room temperature, specifically about 15° C. to about 40° C., more specifically about 20° C. to about 25° C. The ratio of total fenofibric acid to acetonitrile is about 100 to about 300 mg fenofibric acid per milliliter of acetonitrile, specifically about 150 to about 250 mg fenofibric acid per milliliter of acetonitrile, and yet more specifically about 200 to about 225 mg fenofibric acid per milliliter of acetonitrile.

Characterization and determination of the extent, if any, of conversion of fenofibric acid between the crystalline forms can be determined using analytical techniques known in the art, including x-ray powder diffraction (XRPD) analysis, single crystal x-ray diffraction (XRD), Differential Scanning calorimetry (DSC), Raman spectroscopy, Fourier Transform Infrared Spectroscopy (FTIR), Thermo Gravimetric Analysis (TGA), and the like.

Fenofibric acid Form A exhibits a characteristic XRPD pattern peak at 15.4 degrees 2-theta. Form A exhibits characteristic bands at 1647, 1239, 1211, 1115, 859, 845, 770, 657, 510, and 473 cm⁻¹ in its Raman spectrum.

Fenofibric acid Form B exhibits characteristic XRPD pattern peaks at 7.7, 7.9, 17.4 and 24.5±0.2 degrees 2-theta. Form B exhibiting characteristic bands at 1632, 1325, 1259, 1158, 835, 827, 642, and 567 cm⁻¹ in its Raman spectrum.

In one embodiment, the fenofibric acid Form A is substantially free of any other fenofibric acid polymorph.

In one embodiment, the fenofibric acid Form B is substantially free of any other fenofibric acid polymorph.

“Substantially free of other polymorphs” means a material comprises no other polymorphic forms as confirmed by visual inspection as per the aforementioned analytical techniques.

In one embodiment, the fenofibric acid Form A exhibits characteristic peaks of a single polymorph in an X-ray powder diffraction pattern of the material with no observable characteristic peaks of any other polymorph.

In one embodiment, the fenofibric acid Form B exhibits characteristic peaks of a single polymorph in an X-ray powder diffraction pattern of the material with no observable characteristic peaks of any other polymorph.

Also disclosed herein are pharmaceutical compositions comprising the fenofibric acid polymorphs prepared herein.

Solid dosage forms for oral administration include, but are not limited to, capsules, tablets, powders, and granules. In such solid dosage forms, the amorphous dispersion may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules and tablets, the dosage forms may also comprise buffering agents.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, etc., that can provide an oral dosage form alone or when combined with other subunits.

The compositions can be immediate-release forms or controlled-release forms.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release pellets and controlled-release pellets. The immediate-release portion of a combination dosage form may be referred to as a loading dose.

Certain compositions described herein may be “coated”. The coating may be a suitable coating, such as, a functional or a non-functional coating, or multiple functional or non-functional coatings. By “functional coating” is meant to include a coating that modifies the release properties of the total composition, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

The fenofibric acid polymorphs and compositions prepared therefrom are useful in treating conditions such as hypercholesterolemia, hypertriglyceridemia, cardiovascular disorders, coronary heart disease, and peripheral vascular disease (including symptomatic carotid artery disease). The fenofibric acid polymorphs and compositions can be used as adjunctive therapy to diet for the reduction of LDL-C, total-C, triglycerides, and Apo B in adult patients with primary hypercholesterolemia or mixed dyslipidemia (Fredrickson Types Ia and IIb). The fenofibric acid polymorphs and compositions can also be used as adjunctive therapy to diet for treatment of adult patients with hypertriglyceridemia (Fredrickson Types IV and V hyperlipidemia). Markedly elevated levels of serum tryglycerides (e.g., >2000 mg/dL) may increase the risk of developing pancreatitis. The fenofibric acid polymorphs and compositions can also be used for other indications where lipid regulating agents are typically used.

The following examples further illustrate the invention but, of course, should not be construed as in any way limiting its scope.

### EXAMPLES

### Example 1

### Preparation of Fenofibric Acid Polymorph Form A and Form B, Crystallization

Preparation of fenofibric acid polymorphs from single solvent or binary solvent crystallization is achieved according to the conditions shown in Table 1 below.

TABLE 1

| Material | Primary | | | | |
|---|---|---|---|---|---|
| Amount | Solvent | | Anti-solvent | | Form |
| (mg) | (mL) | | (mL) | | (XRPD/Raman) |
| 13.0 | DCM | 0.5 | — | — | A |
| 9.5 | DCM | 0.5 | water | 7.0 | A |
| 12.5 | DCM | 0.5 | heptane | 0.5 | A |
| 15.7 | DCM | 0.5 | c-hexane | 0.5 | A |
| 11.6 | benzene | 3.2 | — | — | B |
| 9.1 | benzene | 3.0 | water | 5.0 | B |
| 9.2 | benzene | 3.0 | heptane | 4.0 | A |
| 11.1 | benzene | 3.0 | c-hexane | 5.0 | A |
| 14.9 | toluene | 7.0 | — | — | B |
| 15.2 | toluene | 9.0 | water | 11.0 | B |
| 15.5 | toluene | 9.0 | heptane | 13.0 | A |
| 13.0 | toluene | 9.0 | c-hexane | 13.0 | B |
| 15.1 | xylenes | 5.0 | — | — | B |
| 10.9 | xylenes | 7.0 | water | 13.0 | B |
| 11.3 | xylenes | 7.0 | heptane | 12.0 | A |
| 12.5 | xylenes | 7.0 | c-hexane | 12.0 | A |

— Single solvent crystallizations

The crystallizations are performed in vials using primary solvents which exhibited moderate to low solubility of the fenofibric acid Form A. About 9-15 milligrams (mg) of fenofibric acid Form A is dissolved in select solvents at 50° C. (Table 1). For the binary solvent crystallizations, anti-solvents including water, heptane or c-hexane are then slowly added until precipitation is observed or until the maximum volume capacity of the vial is reached. The resulting mixtures or solutions are then stirred for five minutes and then cooled rapidly to 0° C. in a refrigerator. If precipitation is observed, the solids are filtered and dried overnight under vacuum at ambient temperature. If no precipitation is observed, the solvent(s) are evaporated under a gentle flow of nitrogen and the residue dried overnight under vacuum.

All solids are analyzed by XRPD and Raman spectroscopy under the following conditions:

The XRPD patterns are obtained with a Shimadzu XRD-6000 according to the following conditions:
Samples for x-ray powder diffraction (XRPD) are analyzed “as is”. Samples are placed on Si zero-return ultra-micro sample holders and analyzed using the following conditions:

| | |
|---|---|
| X-ray tube: | Cu Kα, 40 kV, 40 mA |
| Slits | |
| Divergence Slit | 1.00 deg |
| Scatter Slit | 1.00 deg |
| Receiving Slit | 0.30 mm |
| Scanning | |
| Scan Range | 3.0-45.0 deg |
| Scan Mode | Continuous |
| Step Size | 0.04° |
| Scan Rate | 2°/min |

Raman spectra are obtained using a Kaiser RXN1 Raman Macroscope according to the following conditions:
Raman source: 785 nm laser
Spot Size: 1.2 mm
Single Exposure time: 12 seconds
Co-additions: 16
Enabled Exposure options: Cosmic Ray filtering, Dark subtraction, Intensity calibration

Varying the cooling rate for the crystallization is explored for the crystallization process. Approximately 30 mg of fenofibric acid Form A is dissolved to form a clear solution in a minimal amount of primary solvent at 55° C. The clear solution is passed through a Millipore Millex-HV 0.45 μM syringe filter into a pre-heated vial. For the single solvent crystallizations, the vials are allowed to stir using a magnetic stir bar at 55° C. for 10 minutes and either placed in a refrigerator at 0° C. (rapid cooling, Table 2) or cooled at a rate of 20° C. per hour to room temperature (slow cooling, Table 3). Following cooling, the vials are allowed to stand for 16 hours. For the binary solvent crystallizations, an anti-solvent (water, heptane, or c-hexane) is slowly added to promote precipitation. The vials are then stirred and rapidly cooled using the procedures described for the single solvent crystallizations. If precipitation is observed, the solids are filtered and dried under vacuum (30 inches Hg) at ambient temperature. If no precipitation occurs, the solvent(s) are evaporated under a gentle flow of nitrogen and the residue dried overnight under vacuum (30 inches Hg) at room temperature. The resulting solids are analyzed by XRPD or Raman spectroscopy.

TABLE 2

| Material | Primary | | | | |
|---|---|---|---|---|---|
| Amount | Solvent | | Anti-solvent | | Form |
| (mg) | (mL) | | (mL) | | (XRPD/Raman) |
| 32.1 | MeOH | 3.3 | — | — | A |
| 30.1 | Acetone | 1.5 | — | — | A |
| 28.2 | t-AmOH | 3.3 | — | — | A + peaks |
| 34.1 | MEK | 5.2 | — | — | A + peaks |
| 32.6 | DMA | 1.5 | — | — | A + peaks |
| 34.2 | toluene | 14.25 | — | — | A + B |
| 31.6 | DME | 5.2 | Water | 7.5 | A |
| 30.3 | IPA | 5.2 | Water | 7.5 | A |
| 34.5 | dioxane | 7.1 | Water | 7.5 | A |
| 32.2 | EtOAc | 7.1 | Water | 7.5 | B |
| 27.3 | DMA | 5.2 | Water | 7.5 | A |
| 30.9 | MeOH | 3.3 | Water | 7.5 | A |
| 34.1 | EtOH | 3.3 | Water | 7.5 | A |
| 29.8 | Acetone | 1.5 | Water | 7.5 | A + B |
| 30 | MeCN | 1.5 | Water | 7.5 | A |
| 31.3 | Dioxane | 3.3 | Heptane | 7.5 | B |
| 33.8 | Benzene | 9 | Heptane | 12 | A |
| 33.3 | toluene | 18 | Heptane | 26 | A |
| 30.4 | MeOH | 7.1 | Heptane | 7.5 | A |
| 32.9 | DMSO | 1.5 | Heptane | 7.5 | A |
| 30.4 | DMA | 1.5 | Heptane | 7.5 | A |
| 28.6 | DCM | 1 | c-hexane | 1 | A |
| 30.8 | toluene | 18 | c-hexane | 26 | A |

— Single solvent crystallizations

TABLE 3

| Material | | | | | |
|---|---|---|---|---|---|
| Amount | Primary Solvent | | | | Form |
| (mg) | (mL) | | Anti-solvent (mL) | | (XRPD/Raman) |
| 29.2 | MeOH | 3.3 | — | — | A |
| 31.8 | Acetone | 1.5 | — | — | A |
| 33.1 | t-AmOH | 3.3 | — | — | B |
| 28.9 | MEK | 5.2 | — | — | B |
| 29.1 | DMA | 1.5 | — | — | A + B |
| 27 | toluene | 14 | — | — | A + peaks |
| 33.3 | DME | 5.2 | Water | 7.5 | A |
| 27.7 | IPA | 5.2 | Water | 7.5 | A |
| 31.5 | dioxane | 7.1 | Water | 7.5 | A + B |
| 33.2 | EtOAc | 7.1 | Water | 7.5 | A + peaks |
| 29.9 | DMA | 5.2 | Water | 7.5 | A |
| 28.5 | MeOH | 3.3 | Water | 7.5 | A |
| 27.8 | EtOH | 3.3 | Water | 7.5 | A + B |
| 35 | Acetone | 1.5 | Water | 7.5 | A + B |
| 30.5 | MeCN | 1.5 | Water | 7.5 | A + peaks |
| 26.7 | Dioxane | 3.3 | Heptane | 7.5 | B |
| 32.1 | Benzene | 9 | Heptane | 12 | A |
| 27.8 | toluene | 18 | Heptane | 26 | A |
| 27.4 | MeOH | 7.1 | Heptane | 7.5 | A |
| 32.5 | DMSO | 1.5 | Heptane | 7.5 | A |
| 34.5 | DMA | 1.5 | Heptane | 7.5 | A |
| 29.3 | DCM | 1 | c-hexane | 1 | A + peaks |
| 30.8 | toluene | 18 | c-hexane | 26 | A |

— Single solvent crystallizations

Larger scale crystallizations are performed to prepare fenofibric acid Form B. For the single solvent crystallization, fenofibric acid is dissolved in MEK at 55° C. as indicated in Table 4. The clear solution is stirred between 10-30 minutes either in an oil bath set at 55° C. or at an internal batch temperature of 55° C. The respective oil bath or batch temperature is slowly cooled at a rate of 20° C. per hour to ambient temperature for 3-48 hours. The solvent is then evaporated with a flow of nitrogen over 12 hours and further dried at ambient temperature under 30 inches Hg. Following isolation the solids are analyzed by XRPD or Raman.

For the binary solvent crystallization of Form B, fenofibric acid is dissolved in a primary solvent (EtOAc or dioxane) at 55° C. as indicated in Table 4. The clear solution is stirred between 10-30 minutes in an oil bath set at 55° C. The appropriate anti-solvent is added with no precipitation observed and immediately placed into a 0° C. refrigerator for 16 hours to crash cool. The solvent is then evaporated with a flow of nitrogen over 24-48 hours and further dried at ambient temperature under 30 inches Hg. Following isolation the solids are analyzed by XRPD or Raman.

TABLE 4

| Material | Primary | | | | Stirring | |
|---|---|---|---|---|---|---|
| Amt/Form | Solvent | | Anti-solvent | | time | XRPD/Raman |
| (mg) | (mL) | | (mL) | | (hrs) | (Form) |
| 501/A | MEK | 81 | — | — | 16 | B/A + B |
| 500/A | EtOAc | 110 | Water | 116 | 16 | A + B |
| 500/A | Dioxane | 51 | Heptane | 116 | 16 | A |
| 338/A + B | MEK | 55 | — | — | 16 | B |
| 1000/A | MEK | 162 | — | — | 3 | A + B |
| 825/A + B | MEK | 134 | — | — | 48 | A |
| 733/A | MEK | 119 | — | — | 16 | A |
| 669/A* | MeCN | 3 | — | — | 216 | B |

— Single solvent crystallizations
All lots were isolated by evaporation
*Slurry of Form A seeded with about 6 wt % Form B

### Example 2

### Preparation of Fenofibric Acid Form B by Seeded, Slurrying Method

Form B is prepared by a seeded slurry. Approximately 700 mg of fenofibric acid Form A is slurried with 3-10 wt % Form B in 3 mL of acetonitrile by stirring for up to nine days (216 hours) at room temperature. Following isolation the solids are analyzed by XRPD or Raman.

### Example 3

### Preparation of Fenofibric Acid Polymorphs via Vapor Diffusion

Approximately 13-16 mg of Form A is weighed into individual vials and then dissolved in a minimal amount of primary solvent (dioxane, acetone, DCM or DMA) at ambient conditions as shown in Table 5. The uncapped vials are then placed in a chamber containing a layer of miscible anti-solvent (heptane, water or c-hexane). The chambers are then sealed and allowed to equilibrate at room temperature for up to three days. Solids afforded in all of the chambers are analyzed by XRPD or Raman. As shown, either polymorphic form can be formed depending upon the solvent system chosen.

TABLE 5

| Fenofibric | | | | | | |
|---|---|---|---|---|---|---|
| Acid | Primary | | | | | Form |
| Form A | Solvent | | Anti- | | Precipitation | (XRPD/ |
| (mg) | (mL) | | Solvent | Time | (mg) | Raman) |
| 13.4 | Dioxane | 0.2 | Heptane | 2 Days | Yes* | A |
| 13.9 | Acetone | 0.6 | Water | 1 Day | Yes* | A |
| 14.6 | DMA | 0.4 | Water | 3 Days | 5.9 | A |
| 16.3 | DCM | 7.0 | c-Hex | 2 Days | Yes* | B |

*Primary solvent evaporated in chamber

### Example 4

### Preparation of Fenofibric Acid Polymorphs via Lyophilization

Approximately 20-30 mg of fenofibric acid Form A is added to individual 30 mL amber bottles. Due to the limited solubility of Form A in water, binary solvent mixtures employing water are used to encourage dissolution as shown in Table 6. In addition, a slurry of Form A in water is prepared. Each vial is then stored at −20° C. or −70° C. in an effort to freeze each sample prior to lyophilization. The frozen samples are then lyophilized over a period of 1-3 days under vacuum (10⁻¹ mbar) at −50° C. Upon removal of the solvents, the isolated solids are analyzed by XRPD or Raman. As shown, either polymorphic form or a mixture of Form A and Form B can be isolated depending upon the solvent system chosen.

TABLE 6

| | | | Form |
|---|---|---|---|
| Starting Form | Solvent composition | Time (days) | (XRPD/Raman) |
| A | 100% water | 2 | A |
| A | 68:32 (Water:MeCN) | 1 | B |
| A | 89:11 (Water:IPA) | 3 | A + B |
| A | 89:11 (Water:MeOH) | 1 | A |

### Example 5

### Fenofibric Acid Polymorph Slurry Study

Approximately 30-45 mg in combination of Form B and Form A are weighed to an 8-mL vial outfitted with a stir bar. Solvent (water, toluene, and 1:1 water/IPA, DCM or MeCN) in the amount of 1 mL is added. The samples are sealed with a Teflon cap and parafilm and then placed on a magnetic stirrer at room temperature or 45° C. An aliquot is then sampled after 7 days and 21 days for determination of form by XRPD or Raman (Table 7).

TABLE 7

| Finofibric | | | | | |
|---|---|---|---|---|---|
| Acid | | Initial | Temp | Form, 7 Days | Form, 21 Days |
| (mg) | Solvent (1 mL) | Form | (° C.) | (XRPD/Raman) | (XRPD/Raman) |
| 31.0* | Water | A | RT | A | Diffuse |
| 33.7 | Toluene | A | RT | A | A |
| 33.6 | (1:1) Water/IPA | A | RT | A | A |
| 34.9 | DCM | A | RT | A | A |
| 34.5 | MeCN | A | RT | A | A |
| 33.8* | Water | A | 45 | A | A |
| 32.7 | Toluene | A | 45 | A | A |
| 33.4 | (1:1) Water/IPA | A | 45 | A | A |
| 34.4 | DCM | A | 45 | A + B | A + B |
| 43.3 | MeCN | A | 45 | A | A |
| 30.5* | Water | B | RT | B | B |
| 31.2 | Toluene | B | RT | B | B |
| 31.0 | (1:1) Water/IPA | B | RT | B | B |
| 32.2 | DCM | B | RT | B | B |
| 30.1 | MeCN | B | RT | B | B |
| 32.6* | Water | B | 45 | A | A |
| 31.4 | Toluene | B | 45 | B | B |
| 33.3 | (1:1) Water/IPA | B | 45 | A | A |
| 29.8 | DCM | B | 45 | A + B | A + B |
| 41.3 | MeCN | B | 45 | A | A |
| 15.7 + 16.8* | Water | A + B | RT | A + B | B |
| 16.3 + 16.6 | Toluene | A + B | RT | B | B |
| 16.0 + 16.8 | (1:1) Water/IPA | A + B | RT | B | B |
| 15.1 + 17.6 | DCM | A + B | RT | B | B |
| 15.5 + 15.5 | MeCN | A + B | RT | B | B |
| 17.0 + 15.7* | Water | A + B | 45 | A | A |
| 16.7 + 17.3 | Toluene | A + B | 45 | A | A + B |
| 15.4 + 15.7 | (1:1) Water/IPA | A + B | 45 | A | A |
| 17.0 + 18.2 | DCM | A + B | 45 | A | A |
| 22.1 + 21.3 | MeCN | A + B | 45 | A | A |

*= Material did not wet upon stirring

### Example 6

### Preparation of Fenofibric Acid Form A from Form B, Grinding Study

Approximately 50 mg of Form B is ground for 30 seconds using a ball mill and the resulting material is analyzed by XRPD. Form A is observed.

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing fenofibric acid Form B, comprising:
crystallizing fenofibric acid from a crystallization solution comprising fenofibric acid and a solvent system comprising
i) ethyl acetate;
ii) methyl ethyl ketone;
iii) ethyl acetate as a primary solvent and water as an anti-solvent;
iv) dioxane as a primary solvent and heptane as an anti-solvent; or
v) tert-amyl alcohol.

2. The method of claim 1, wherein the solvent system is methyl ethyl ketone.

3. The method of claim 1, wherein the crystallization solution during crystallization is about −10 to about 30° C.

4. The method of claim 1, wherein the crystallization solution has an initial temperature when prepared that is greater than the crystallization solution during crystallization.

5. The method of claim 4, wherein the crystallization solution is allowed to cool from the initial temperature to a final temperature during crystallization at a rate of about 1 to about 30° C. per hour.

6. The method of claim 4, wherein the crystallization solution is prepared with heating and subsequently subjected to a temperature that is the final temperature during crystallization without allowing for the crystallization solution to cool gradually.

7. The method of claim 1, wherein fenofibric acid Form B is substantially free of fenofibric acid Form A.

8. A method of preparing fenofibric acid Form A, comprising:
crystallizing fenofibric acid from a crystallization solution comprising fenofibric acid and a solvent system comprising
i) dichloromethane;
ii) methanol;
iii) acetone;
iv) dichloromethane as a primary solvent and water, heptane, or c-hexane as an anti-solvent;
v) benzene, toluene, or xylenes as a primary solvent and heptane or c-hexane as an anti-solvent;
v) 1,2-dimethoxyethane, isopropanol, dioxane, dimethylacetamide, methanol, ethanol, or acetonitrile as a primary solvent and water as an anti-solvent; or
vi) methanol, dimethyl sulfoxide, or dimethylacetamide as a primary solvent and heptane as an anti-solvent.

9. The method of claim 8, wherein the solvent system is dichloromethane.

10. The method of claim 9, wherein the crystallization solution during crystallization is about −10 to about 30° C.

11. The method of claim 9, wherein the crystallization solution has an initial temperature when prepared that is greater than the crystallization solution during crystallization.

12. The method of claim 11, wherein the crystallization solution is allowed to cool from the initial temperature to a final temperature during crystallization at a rate of about 1 to about 30° C. per hour.

13. The method of claim 11, wherein the crystallization solution is prepared with heating and subsequently subjected to a temperature that is the final temperature during crystallization without allowing for the crystallization solution to cool gradually.

14. The method of claim 8, wherein fenofibric acid Form A is substantially free of fenofibric acid Form B.

15. A method of preparing fenofibric acid Form B, comprising:
slurrying fenofibric acid Form A seeded with fenofibric acid Form B in acetonitrile at about 15° C. to about 40° C.; or
slurrying a mixture of Form A and Form B in water, toluene, water/isopropyl alcohol, dichloromethane, or acetonitrile at about 15° C. to about 40° C.

16. The method of claim 15, wherein the ratio of total fenofibric acid to acetonitrile is about 100 to about 300 mg fenofibric acid per milliliter of acetonitrile.

17. The method of claim 15, wherein fenofibric acid Form B is substantially free of fenofibric acid Form A.

18. A method of preparing fenofibric acid Form A, comprising:
slurrying fenofibric acid Form B in water, acetonitrile, or 1:1 water/isopropyl alcohol at about 30° C. to about 50° C.; or
slurrying a mixture of Form A and Form B in water, toluene, 1:1 water/isopropyl alcohol, dichloromethane, or acetonitrile at about 30° C. to about 50° C.

19. The method of claim 18, wherein fenofibric acid Form A is substantially free of fenofibric acid Form B.

20. A method of preparing fenofibric acid Form A, comprising:
lyophilizing a mixture of fenofibric acid and water or a mixture of fenofibric acid, water and methanol.

21. The method of claim 20, wherein fenofibric acid Form A is substantially free of fenofibric acid Form B.

22. A method of preparing fenofibric acid Form B, comprising:
lyophilizing a mixture of fenofibric acid, water, and acetonitrile.

23. The method of claim 22, wherein fenofibric acid Form B is substantially free of fenofibric acid Form A.

24. A method of preparing fenofibric acid Form A, comprising:
grinding fenofibric acid Form B or a combination comprising fenofibric acid Form B and Form A under high shear conditions.

25. The method of claim 24, wherein the grinding is performed in a ball mill.

26. A method of preparing fenofibric acid Form A or Form B, comprising:
preparing fenofibric acid Form A or Form B by vapor diffusion.
